(19) United States
(12) Patent Application Publication
(10) Pub. No.: US 2024/0346405 A1
(43) Pub. Date: Oct. 17, 2024

(54) MEDICAL INFORMATION PROCESSING APPARATUS, MEDICAL INFORMATION PROCESSING METHOD, AND STORAGE MEDIUM

(71) Applicant: CANON MEDICAL SYSTEMS CORPORATION, Tochigi (JP)

(72) Inventors: Kei YAMAJI, Nasushiobara (JP); Masashi YOSHIDA, Nasushiobara (JP); Kouji OTA, Nasushiobara (JP)

(73) Assignee: CANON MEDICAL SYSTEMS CORPORATION, Tochigi (JP)

(21) Appl. No.: 18/632,370

(22) Filed: Apr. 11, 2024

(30) Foreign Application Priority Data
Apr. 11, 2023 (JP) 2023-064052

(51) Int. Cl. G06Q 10/0631; G16H 40/20

(52) U.S. Cl. CPC G06Q 10/063112; G16H 40/20

(57) ABSTRACT

A medical information processing apparatus according to an embodiment includes processing circuitry. The processing circuitry detects an event that requires an operation by an operator in a medical-related work performed by an apparatus connected to a network. The processing circuitry determines at least one operator who can respond to the event among operators who operate terminal apparatuses connected to the network. The processing circuitry notifies the terminal apparatus operated by the at least one operator of the occurrence of the event.

### CROSS-REFERENCE TO RELATED APPLICATIONS

This application is based upon and claims the benefit of priority from Japanese Patent Application No. 2023-064052, filed on Apr. 11, 2023; the entire contents of which are incorporated herein by reference.

### FIELD

Embodiments disclosed herein relate generally to a medical information processing apparatus, a medical information processing method, and a storage medium.

### BACKGROUND

In recent years, reforms to the way physicians and medical professionals work have been required, and with overtime caps coming into effect for physicians in FY2024, an urgent need exists to improve the efficiency of their work. To improve the efficiency of the work, emphasis is being placed on the use of information and communication technology (ICT), robots, and the like.

### BRIEF DESCRIPTION OF THE DRAWINGS

FIG. 1 is a block diagram illustrating an example of a system including a medical information processing apparatus according to a first embodiment;

FIG. 2 is a block diagram illustrating an example of the configuration of the medical information processing apparatus according to the first embodiment;

FIG. 3 is a flowchart illustrating an example of the processing procedure of the medical information processing apparatus according to the first embodiment;

FIG. 4 is a diagram illustrating an example of a participant table according to the first embodiment;

FIG. 5 is a diagram for explaining event notification according to the first embodiment;

FIG. 6A is a diagram for explaining an example of displaying event details according to the first embodiment;

FIG. 6B is a diagram for explaining an example of displaying event details according to the first embodiment;

FIG. 7A is a diagram illustrating an example of an operation screen according to the first embodiment;

FIG. 7B is a diagram illustrating an example of an operation screen according to the first embodiment;

FIG. 8 is a diagram illustrating an example of an operation screen according to the first embodiment;

FIG. 9 is a diagram illustrating an example of an operation screen according to the first embodiment; and

FIG. 10 is a diagram for explaining a process according to an operation by an operator according to the first embodiment.

### DETAILED DESCRIPTION

A medical information processing apparatus according to an embodiment includes processing circuitry. The processing circuitry is configured to detect an event that requires an operation by an operator in a medical-related work performed by an apparatus connected to a network. The processing circuitry is configured to determine at least one operator who can respond to the event among operators who operate terminal apparatuses connected to the network. The processing circuitry is configured to notify the terminal apparatus operated by the at least one operator of the occurrence of the event.

Embodiments of a medical information processing apparatus, a medical information processing method, and a storage medium are described in detail below with reference to the drawings. Note that the medical information processing apparatus, the medical information processing method, and the storage medium according to the present application are not limited by the following embodiments.

### First Embodiment

As described above, high efficiency of work is required in today's medical site. To this end, for example, one operator (physician or laboratory technician) can remotely operate a plurality of medical image diagnostic apparatuses to improve the efficiency of work. However, when one operator remotely operates the medical image diagnostic apparatuses, work may stop once the operator's operations are delayed. In addition, when one operator remotely operates the medical image diagnostic apparatuses, the operator may confuse and erroneously select and use the apparatuses. The medical information processing apparatus according to the present embodiment can appropriately improve the efficiency of work by requesting an operation to an operator who can respond to the request at the timing when operations by the operator are required.

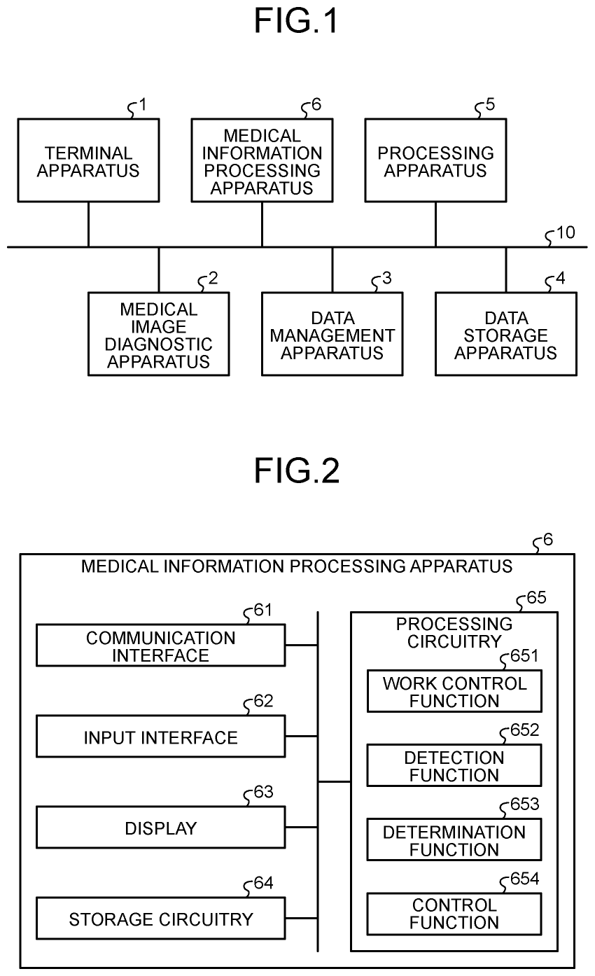

FIG. 1 is a block diagram illustrating an example of a system including the medical information processing apparatus according to the first embodiment. For example, as illustrated in FIG. 1, a medical information processing apparatus 6 according to the present embodiment is included in a system in which a terminal apparatus 1, a medical image diagnostic apparatus 2, a data management apparatus 3, a data storage apparatus 4, and a processing apparatus 5 are connected via a network 10, and can communicably connected to various apparatuses via the network 10. The network 10 includes, for example, a local area network (LAN) and a wide area network (WAN) installed in a hospital. Note that various other apparatuses and systems may be connected to the network 10 illustrated in FIG. 1.

The terminal apparatus 1 is an apparatus operated by a physician or a laboratory technician working in a hospital. For example, the terminal apparatus 1 is implemented by a personal computer (PC), a tablet PC, a personal digital assistant (PDA), a mobile phone, or the like. The terminal apparatus 1 displays various information received from the medical information processing apparatus 6 on its own display and receives various operations via its own input interface. For example, the terminal apparatus 1 receives input operations for orders for various examinations, examination results, diagnostic information, and the like, and transmits the input information to the medical information processing apparatus 6. Although FIG. 1 illustrates a single terminal apparatus 1, a plurality of terminal apparatuses 1 are actually connected to the network 10.

The medical image diagnostic apparatus 2 takes images of a subject and generates medical images. The medical image diagnostic apparatus 2 also transmits the generated medical images to various apparatuses on the network. For example, the medical image diagnostic apparatus 2 includes an X-ray computed tomography (CT) apparatus, a magnetic resonance imaging (MRI) apparatus, an X-ray diagnostic apparatus, an ultrasound diagnostic apparatus, a single photon emission computed tomography (SPECT) apparatus, a positron emission computed tomography (PET) apparatus, and the like. Although FIG. 1 illustrates a single medical image diagnostic apparatus 2, various medical image diagnostic apparatuses 2 described above are actually connected to the network 10.

The data management apparatus 3 manages medical information of the subject. Specifically, the data management apparatus 3 links and centrally manages raw data collected by imaging the subject, medical images obtained by reconstructing the raw data, analysis results obtained by analyzing the medical images, reports indicating the results of reading the medical images, and the like. The actual medical information data (the above-described raw data, medical images, analysis results, reports, and the like) are stored by the data storage apparatus 4. That is, the data management apparatus 3 links and manages information on the medical information data described above (date and time of collection, data storage location, and the like). For example, the data management apparatus 3 is implemented by computer equipment such as servers and workstations.

The data storage apparatus 4 stores the medical information of the subject. Specifically, the data storage apparatus 4 receives medical information data (the raw data, the medical images, the analysis results, the reports, and the like) from the terminal apparatus 1, the medical image diagnostic apparatus 2, and the processing apparatus 5 via the network 10, and stores the received medical information data in its own storage circuitry. For example, the data storage apparatus 4 is implemented by a picture archiving and communication system (PACS), a hospital information system (HIS), a radiology information system (RIS), or the like.

The processing apparatus 5 reconstructs medical images on the basis of data collected by the medical image diagnostic apparatus 2, and performs various analysis processes based on the medical images. For example, the processing apparatus 5 is implemented by computer equipment such as servers and workstations.

The medical information processing apparatus 6 supports medical professionals such as physicians and laboratory technicians by performing and managing their work in the system. Specifically, on the basis of the medical information managed by the data management apparatus 3, the medical information processing apparatus 6 constructs a workflow suitable for the examination orders received from the terminal apparatus 1, and provides the workflow to the medical professional. Subsequently, the medical information processing apparatus 6 controls the apparatuses on the network 10 to perform various works based on the constructed workflow under the approval of the medical professional. For example, the medical information processing apparatus 6 collects raw data by controlling the medical image diagnostic apparatus 2. Subsequently, the medical information processing apparatus 6 transmits the collected raw data to the processing apparatus 5, so that the processing apparatus 5 reconstructs the medical images and performs various analysis processes. The medical information processing apparatus 6 also transmits the medical images and analysis results to the terminal apparatus 1 to facilitate reading and report writing.

The medical information processing apparatus 6 monitors the progress of work being performed on the system and performs control so that the workflow proceeds smoothly. That is, the medical information processing apparatus 6 performs various controls so that the workflow proceeds automatically. In situations where judgment (and operation) by an operator (medical professional) is required in work, the medical information processing apparatus 6 can provide the terminal apparatus 1 with an operation screen for inputting judgment results. In other words, the medical information processing apparatus 6 can perform control so that works other than those requiring judgment (and operation) by the operator (medical professional) proceed automatically in works being performed on the system.

Subsequently, the medical information processing apparatus 6 according to the present embodiment performs various processes for appropriately improving the efficiency of work. Specifically, the medical information processing apparatus 6 detects an event that requires an operation by an operator in works being performed on the system, specifies an operator who can respond to the detected event (operator who has the authority and ability to respond to the event) among operators who operate the terminal apparatuses 1 connected to the system, and notifies the specified operator of the occurrence of the event. The medical information processing apparatus 6 notifies all operators who can respond to the event of the occurrence of the event, and controls the system to receive responses from operators who can respond to the event in terms of time and work at the time the event has occurred, thereby suppressing the occurrence of mistakes and appropriately improving the efficiency of work. For example, the medical information processing apparatus 6 is implemented by computer equipment such as servers and workstations.

FIG. 2 is a block diagram illustrating an example of the configuration of the medical information processing apparatus 6 according to the first embodiment. As illustrated in FIG. 2, the medical information processing apparatus 6 includes a communication interface 61, an input interface 62, a display 63, storage circuitry 64, and processing circuitry 65.

The communication interface 61 controls the transmission and communication of various data transmitted and received between the medical information processing apparatus 6 and each apparatus connected via the network 10. Specifically, the communication interface 61 is connected to the processing circuitry 65, and transmits data received from each apparatus on the network 10 to the processing circuitry 65, or transmits data received from the processing circuitry 65 to each apparatus on the network 10. For example, the communication interface 61 is implemented by a network card, a network adapter, a network interface controller (NIC), or the like.

The input interface 62 receives input operations for various instructions and various information from an operator. Specifically, the input interface 62 is connected to the processing circuitry 65, and converts the input operations received from the operator into electrical signals and transmits the electrical signals to the processing circuitry 65. For example, the input interface 62 is implemented by a trackball, a switch button, a mouse, a keyboard, a touchpad for performing input operations by touching an operation surface, a touchscreen in which a display screen and a touchpad are integrated, a non-contact input interface using an optical sensor, a voice input interface, and the like. In the present specification, the input interface 62 is not limited only to those with physical operating components such as a mouse and a keyboard. For example, an example of the input interface 62 also includes electrical signal processing circuitry that receives electrical signals corresponding to input operations from an external input device installed separately from the device and transmits the electrical signals to control circuitry.

The display 63 displays various information and various data. Specifically, the display 63 is connected to the processing circuitry 65 and displays various information and various data received from the processing circuitry 65. For example, the display 63 is implemented by a liquid crystal display, a cathode ray tube (CRT) display, a touch panel, or the like.

The storage circuitry 64 stores therein various data and various computer programs. Specifically, the storage circuitry 64 is connected to the processing circuitry 65, and stores therein data received from the processing circuitry 65 or reads stored data and transmits the read data to the processing circuitry 65. For example, the storage circuitry 64 is implemented by a semiconductor memory element such as a read-only memory (ROM), a random-access memory (RAM), and a flash memory, a hard disk, an optical disc, or the like. Note that the storage circuitry 64 may be implemented by a cloud computer connected to the medical information processing apparatus 6 via the network 10.

The processing circuitry 65 controls the entire medical information processing apparatus 6. Specifically, the processing circuitry 65 performs and manages works in the system. For example, the processing circuitry 65 performs construction of a workflow based on received orders, control of the medical image diagnostic apparatus 2 and the processing apparatus 5, access to the data management apparatus 3 and the data storage apparatus 4, and the like. The processing circuitry 65 also controls transmission and reception of information to and from the terminal apparatus 1. Note that the processing circuitry 65 can also perform various processes (exchanging data with the apparatuses on the network 10, storing data in the storage circuitry 64, displaying information on the display 63, and the like) according to input operations via the input interface 62.

For example, as illustrated in FIG. 2, in the present embodiment, the processing circuitry 65 of the medical information processing apparatus 6 performs a work control function 651, a detection function 652, a determination function 653, and a control function 654. The processing circuitry 65 is an example of processing circuitry.

The work control function 651 causes an apparatus connected to the network 10 to perform a medical-related work and controls the progress of the work. Specifically, the work control function 651 performs construction of a workflow based on the medical information managed by the data management apparatus 3, control of the medical image diagnostic apparatus 2 and the processing apparatus 5 based on the constructed workflow, and the like. Note that the process performed by the work control function 651 is described in detail later.

The detection function 652 detects an event that requires an operation by an operator in the medical-related work performed by the apparatus connected to the network 10. Specifically, the detection function 652 detects an event that requires judgment and operation by a medical professional in a workflow being performed on the system under the control of the work control function 651. Note that the process performed by the detection function 652 is described in detail later.

The determination function 653 determines at least one operator who can respond to the event among operators who operate the terminal apparatuses 1 connected to the network 10. Specifically, when an event is detected by the detection function 652, the determination function 653 determines at least one operator who can respond to the event at the time the event is detected. Note that the process performed by the determination function 653 is described in detail later.

The control function 654 notifies the terminal apparatus 1 operated by the at least one operator determined by the determination function 653 of the occurrence of the event. The control function 654 also causes the terminal apparatus 1 of the operator who has responded to the notification to display information on details of the event. Note that the process performed by the control function 654 is described in detail later.

The processing circuitry 65 described above is implemented by, for example, a processor. In such a case, the respective processing functions described above are stored in the storage circuitry 64 in the form of computer programs executable by a computer. The processing circuitry 65 reads the computer programs stored in the storage circuitry 64 and executes the read computer programs, thereby implementing functions corresponding to the computer programs. In other words, the processing circuitry 65 in the state of reading the computer programs has each of the processing functions illustrated in FIG. 2.

Note that the processing circuitry 65 may be configured by combining a plurality of independent processors, and the processors may implement respective processing functions by executing respective computer programs. The processing functions of the processing circuitry 65 may be implemented by being appropriately distributed or integrated into single processing circuitry or a plurality of pieces of processing circuitry. Each processing function of the processing circuitry 65 may be implemented by a mixture of hardware such as circuits and software. Although an example in which the computer program corresponding to each processing function is stored in one piece of the storage circuitry 64 is described above, the embodiment is not limited thereto. For example, a computer program corresponding to each processing function may be distributed and stored in a plurality of pieces of storage circuitry, and the processing circuitry 65 may be configured to read each computer program from each piece of the storage circuitry and execute the read computer program. Note that some of the processing functions of the processing circuitry 65 may be implemented by a cloud computer connected to the medical information processing apparatus 6 via the network 10.

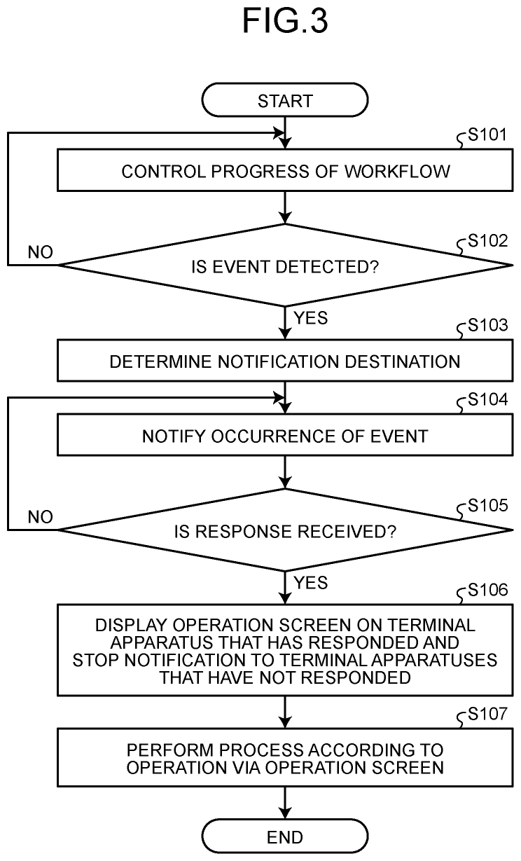

The processing procedure performed by the medical information processing apparatus 6 is described below with reference to FIG. 3, and then details of each process are described. FIG. 3 is a flowchart illustrating an example of the processing procedure of the medical information processing apparatus 6 according to the first embodiment.

For example, as illustrated in FIG. 3, in the present embodiment, the work control function 651 controls the progress of a workflow (step S101). For example, the work control function 651 constructs a workflow based on an order by controlling each apparatus connected to the network 10, and performs various processes in the constructed workflow. This process is implemented, for example, by the processing circuitry 65 that calls a computer program corresponding to the work control function 651 from the storage circuitry 64 and executes the called computer program.

Subsequently, the detection function 652 detects an event that requires judgment and operation by a medical professional in the workflow (step S102). This process is implemented, for example, by the processing circuitry 65 that calls a computer program corresponding to the detection function 652 from the storage circuitry 64 and executes the called computer program.

When the event is detected at step S102 (YES at step S102), the determination function 653 determines a notification destination to be notified of the occurrence of the event (step S103). This process is implemented, for example, by the processing circuitry 65 that calls a computer program corresponding to the determination function 653 from the storage circuitry 64 and executes the called computer program. Note that the workflow is conducted by the work control function 651 until an event is detected at step S102 (NO at step S102).

Subsequently, the control function 654 notifies the terminal apparatus 1 at the determined notification destination of the occurrence of the event (step S104). When a response to the event notification is received (YES at step S105), the control function 654 causes the terminal apparatus 1 that has responded to display an operation screen and stops notifications to terminal apparatuses that have not responded (step S106). Note that the control function 654 continues to perform notification until a response is received (No at step S105). This process is implemented, for example, by the processing circuitry 65 that calls a computer program corresponding to the control function 654 from the storage circuitry 64 and executes the called computer program.

Subsequently, the work control function 651 performs a process according to an operation via the operation screen displayed on the terminal apparatus 1 (step S107). This process is implemented, for example, by the processing circuitry 65 that calls the computer program corresponding to the work control function 651 from the storage circuitry 64 and executes the called computer program.

The following is a detailed description of each process performed by the medical information processing apparatus 6.

### Workflow Control Process

As described at step S101 of FIG. 3, the work control function 651 controls the construction and execution of the workflow. Specifically, on the basis of the medical information of the subject managed by the data management apparatus 3, the work control function 651 constructs a workflow suitable for the order received from the terminal apparatus 1. For example, the work control function 651 acquires, from the data management apparatus 3, past medical information of the subject of the order received from the terminal apparatus 1, medical information corresponding to examination details described in the order, and the like, and constructs a workflow suitable for the order on the basis of the acquired medical information.

In one example, the work control function 651 constructs a workflow by determining, on the basis of the medical information, the type of the medical image diagnostic apparatus 2 that performs a scan, imaging conditions (scan protocol, scan parameters, and the like), reconstruction conditions, type of an analysis process, and the like. Subsequently, the work control function 651 stores the constructed workflow in the storage circuitry 64.

### Event Detection Process

As described at step S102 of FIG. 3, the detection function 652 detects an event that requires judgment and operation by the medical professional in the workflow. For example, the detection function 652 detects operator's approval work in constructing the workflow, confirmation work after a scan is completed, confirmation work of findings in an automatically generated report, confirmation work when a problem occurs during the progress of the workflow, and the like. The event that requires judgment and operation by the medical professional is set in advance, and information on the set event is stored in the storage circuitry 64. That is, the detection function 652 detects the event that requires judgment and operation by the medical professional by referring to the event-related information listed in the storage circuitry 64.

### Notification Destination Determination Process

As described at step S103 of FIG. 3, the determination function 653 determines the notification destination for the event detected by the detection function 652. Specifically, the determination function 653 first specifies the terminal apparatuses 1 connected to the network 10, and determines an operator who can respond to the event among operators who operate the specified terminal apparatuses 1.

In order to specify the terminal apparatuses 1 connected to the network 10, the determination function 653 prepares a participant table for managing the terminal apparatuses 1 connected to the network 10. Specifically, the determination function 653 monitors the connection status of the terminal apparatuses 1 to the network 10, and when a terminal apparatus 1 is connected to the network 10, the determination function 653 acquires information on the connected terminal apparatus 1 and registers the acquired information in the participant table.

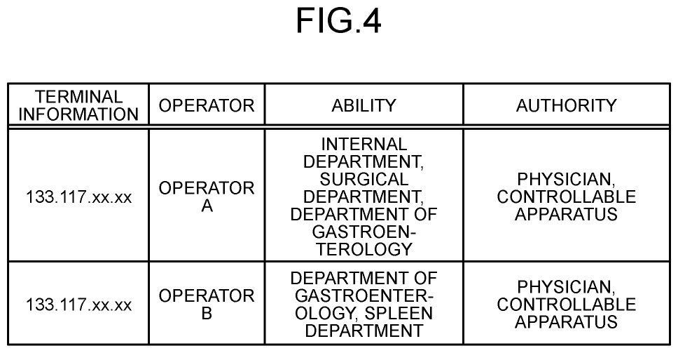

FIG. 4 is a diagram illustrating an example of the participant table according to the first embodiment. For example, as illustrated in FIG. 4, the determination function 653 prepares the participant table that maps “terminal information”, “operator”, “ability”, and “authority”. In FIG. 4, the “terminal information” refers to information for identifying the terminal apparatuses 1 connected to the network 10, and the “operator” refers to information of operators who operate the terminal apparatuses 1 identified by the “terminal information”. In FIG. 4, the “ability” refers to operator's ability information, and the “authority” refers to operator's authority information.

For example, as illustrated in FIG. 4, the determination function 653 registers an address of the terminal apparatus 1 as the “terminal information”, registers a name of the operator as the “operator”, registers information of a clinical department as the “ability”, and registers information on the type of job and a controllable apparatus as the “authority”. The determination function 653 monitors the connection status of the terminal apparatuses 1 to the network 10, and registers the above information, in the participant table, on the terminal apparatuses 1 connected to the network 10. When the connection of a certain terminal apparatus 1 to the network 10 is cut off, the determination function 653 deletes the information of the terminal apparatus 1 from the participant table.

The information illustrated in FIG. 4 is merely an example, and other information may be registered in the participant table. For example, the installation status, version, and the like of an operating system (OS) or software of the terminal apparatus 1 may be registered as the “terminal information”. For example, the presence or absence of diagnostic experience related to an examination, experience with an apparatus, years of service, and the like may be registered as the “ability”.

The determination function 653 specifies the terminal apparatuses 1 connected to the network 10 on the basis of the participant table, and determines the terminal apparatus 1 to be operated by an operator who can respond to an event among the specified terminal apparatuses 1. Specifically, the determination function 653 determines all terminal apparatuses 1 operated by the operators who can respond to details of the event detected by the detection function 652 among the terminal apparatuses 1 registered in the participant table. More specifically, on the basis of the authority information and the ability information of the operators who operate the terminal apparatuses 1 connected to the network 10, the determination function 653 determines the operator who can respond to the event.

For example, when a post-scan confirmation work is detected as the event, the determination function 653 compares examination information included in a workflow, the type of the medical image diagnostic apparatus 2, a site to be scanned, and the like with the ability information and the authority information in the participant table, and determines all operators who can perform the post-scan confirmation work for a scan performed this time.

### Event Notification Process

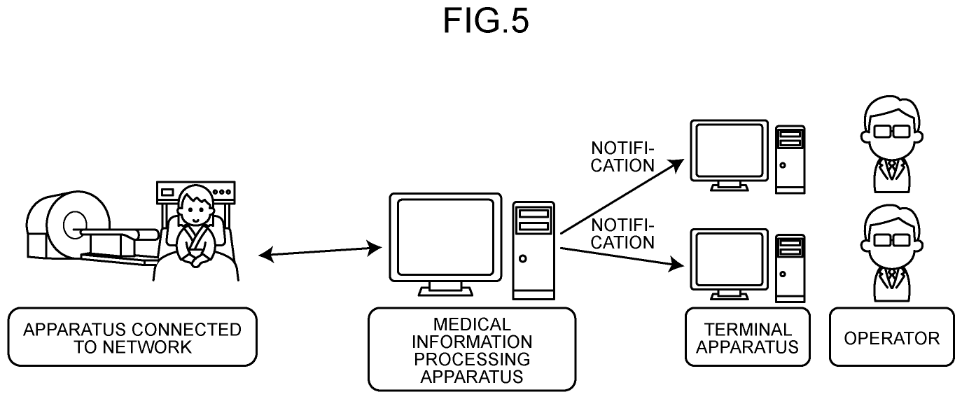

As described at step S104 of FIG. 3, the control function 654 notifies the notification destination (terminal apparatus 1) determined by the determination function 653 of the occurrence of the event. FIG. 5 is a diagram for explaining event notification according to the first embodiment. As illustrated in FIG. 5, the medical information processing apparatus 6 communicates with the apparatuses connected to the network 10, determines the terminal apparatus 1 of an operator who can respond to an event in response to the occurrence of the event, and notifies the determined terminal apparatus 1 of the occurrence of the event. The control function 654 notifies all the terminal apparatuses 1 determined by the determination function 653 of the occurrence of the event.

For example, the control function 654 causes displays of all the terminal apparatuses 1 determined by the determination function 653 to display information indicating that the event has occurred. The operator notified of the occurrence of the event is usually on duty or at work. In this regard, the control function 654 causes the information indicating that the event has occurred to be displayed in a recognizable display format that does not interfere with a work currently being performed.

### Process of Displaying Event Details

As described at steps S105 and S106 of FIG. 3, the control function 654 causes the terminal apparatus 1 of the operator who has responded to the notification of the occurrence of the event to display information on details of the event. Specifically, the control function 654 causes the terminal apparatus of the operator who has responded to the notification to display an operation screen for performing an operation on the event.

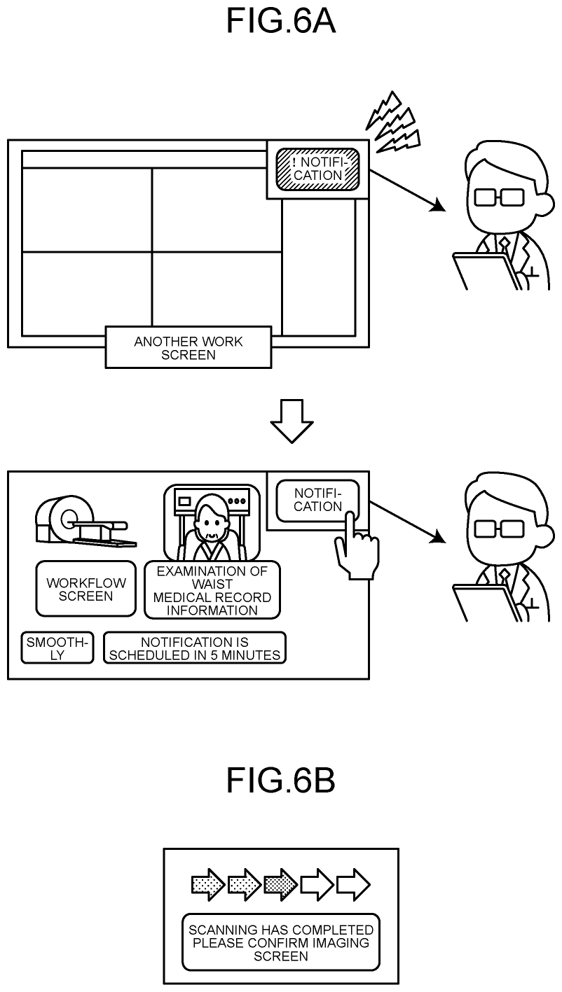

FIGS. 6A and 6B are diagrams for explaining an example of displaying event details according to the first embodiment. For example, as illustrated in an upper diagram in FIG. 6A, the control function 654 causes the displays of all the terminal apparatuses 1 determined by the determination function 653 to display a notification indicating that the event has occurred. That is, the control function 654 causes the terminal apparatus 1 on which a work is being performed on another work screen to display the notification indicating that the event has occurred.

When one of the operators responds to this notification, the control function 654 causes the terminal apparatus 1 operated by the responding operator to display a GUI including a workflow screen, examination and medical record information, progress status, and the like, as illustrated in a lower diagram in FIG. 6A. This allows the responding operator to ascertain the current status in the workflow where the event has occurred.

In the workflow screen, for example, as illustrated in FIG. 6B, the control function 654 enables the display of information that shows details of a work required to be performed by the operator (confirmation of an imaging screen) in a manner that the operator understands at a glance. Note that the control function 654 enables the display of the entire workflow so that details of the current work in the workflow can be seen.

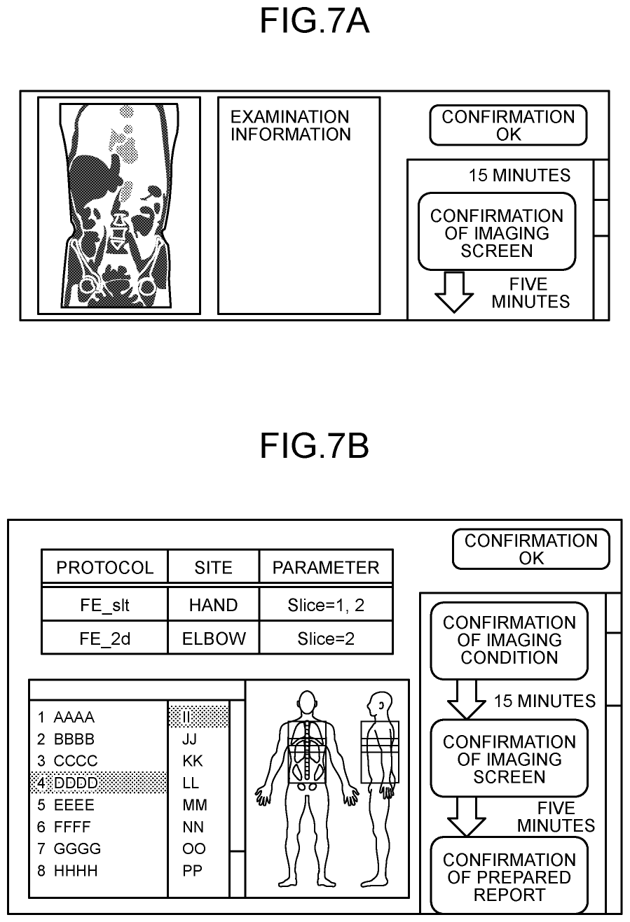

Subsequently, the control function 654 causes the display of the terminal apparatus 1 to display, for example, a GUI illustrated in FIG. 7A, as the confirmation of the imaging screen. For example, as illustrated in FIG. 7A, the control function 654 causes the display of the terminal apparatus 1 to display an operation screen indicating medical images obtained by a scan, examination information, and a workflow, and including a GUI for approval. That is, the control function 654 causes the display of the terminal apparatus 1 to display an operation screen that is easy to confirm details of the work and easy to operate.

In the present embodiment, since details of a work to be performed by an operator is clear from the workflow, an operation screen including only necessary information can be displayed as illustrated in FIG. 7A. This is true for other works as well. For example, when imaging conditions are confirmed, the control function 654 can cause the display of the terminal apparatus 1 to display an operation screen that allows a list of the imaging conditions, set conditions, and a workflow to be checked at a glance, as illustrated in FIG. 7B. FIGS. 7A and 7B are diagrams illustrating an example of an operation screen according to the first embodiment.

The control function 654 can also change display details of the operation screen according to the status of a notification destination. Specifically, the control function 654 changes the display details of the operation screen according to the attributes of an operator who has responded to a notification. For example, the control function 654 refers to the “ability” in the participant table, and changes the display details of the operation screen on the basis of information such as operator's years of service, the presence or absence of an examination-related diagnosis, and experience of an apparatus.

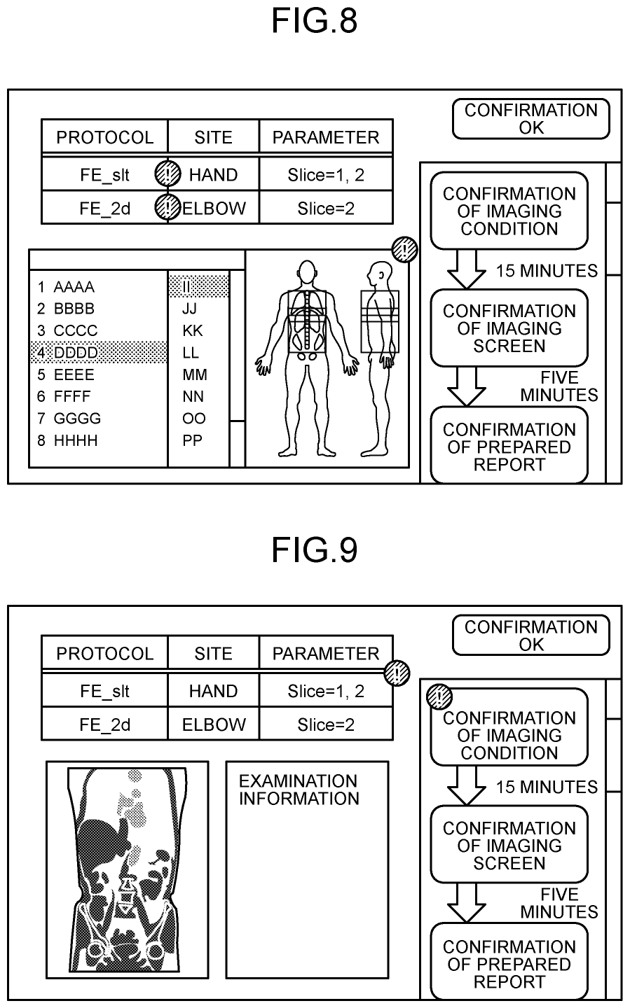

FIG. 8 is a diagram illustrating an example of an operation screen according to the first embodiment. For example, when an operator who has responded to a notification is a medical professional who has worked for a short period of time, is inexperienced in an examination-related diagnosis, or is inexperienced in an apparatus, the control function 654 displays an operation screen with a “!” mark attached to a portion of interest on the operation screen, as illustrated in FIG. 8. The “!” mark is attached with information for guiding points to be confirmed, and by moving a cursor to the “!” mark, the attached information is displayed. That is, when the operator who has responded to the notification is an inexperienced medical professional, the control function 654 can display supportive information.

When the operator who has responded to the notification is an inexperienced medical professional, the control function 654 can also display the operation screen by replacing difficult terms with simple terms. On the other hand, when the operator who has responded to the notification is an experienced medical professional, the control function 654 may also display an easier-to-read operation screen with no unnecessary information without displaying the “!” mark or replacing difficult terms with simple terms.

When the operator who has responded to the notification is an experienced medical professional, the control function 654 can also improve confirmation efficiency by displaying a list in a table. When the operator who has responded to the notification is an inexperienced medical professional, the control function 654 can also provide an easy-to-understand display using illustrations or the like.

For example, even when an operator who performs a post-scan confirmation work is different from an operator who has approved the imaging conditions, the control function 654 can change display details of the operation screen. FIG. 9 is a diagram illustrating an example of an operation screen according to the first embodiment. FIG. 9 illustrates the operation screen for the operator to change the imaging conditions set by the work control function 651 in the approval of the imaging conditions, to perform a scan with the changed imaging conditions, and to perform a post-scan confirmation.

In such a situation, when the operator who performs the post-scan confirmation is different from the operator who has approved (changed) the imaging conditions, the control function 654 displays a “!” mark for the changed conditions, as illustrated in FIG. 9. The “!” mark is attached with details of the change, the reason for the change, information for identifying the operator who has changed the imaging conditions, and the like, and by moving a cursor to the “!” mark, the attached information is displayed.

The control function 654 can also change display details of the operation screen according to the status of the terminal apparatus 1 operated by the operator. For example, when the terminal apparatus 1 operated by the operator who has responded to the notification is a tablet, the control function 654 separately displays display details according to the screen size of the tablet. In one example, the control function 654 can control workflow information and imaging conditions to be displayed in sequence. The control function 654 can also control appropriate display according to the OS or software installed in the terminal apparatus 1 or the version information.

As described above, the control function 654 causes the terminal apparatus 1 operated by the operator who has responded to the notification among the operators notified of the occurrence of the event to display the operation screen for performing an operation on the event.

On the other hand, the control function 654 causes the terminal apparatus 1 operated by an operator (operator who has not responded to the notification) other than the operator who has responded to the notification, among the terminal apparatuses 1 notified of the occurrence of the event, to transition to a state in which it is identifiable that the response to the notification has been executed. For example, the control function 654 stops the notification to the terminal apparatus 1 operated by the operator other than the operator who has responded to the notification. That is, the control function 654 controls the notification to be turned off from the display of the terminal apparatus 1.

Note that the control to make it identifiable that the response to the notification has been executed may include not only turning off the notification from the display of the terminal apparatus 1, but also displaying information indicating that another operator has responded to the notification. The control function 654 can also cause information on details of the event to be displayed on the display of the terminal apparatus 1 operated by an operator who has not responded to the notification. That is, the control function 654 can perform a control so that even the operator who has not responded to the notification can confirm details of the event.

### Process According to Operation

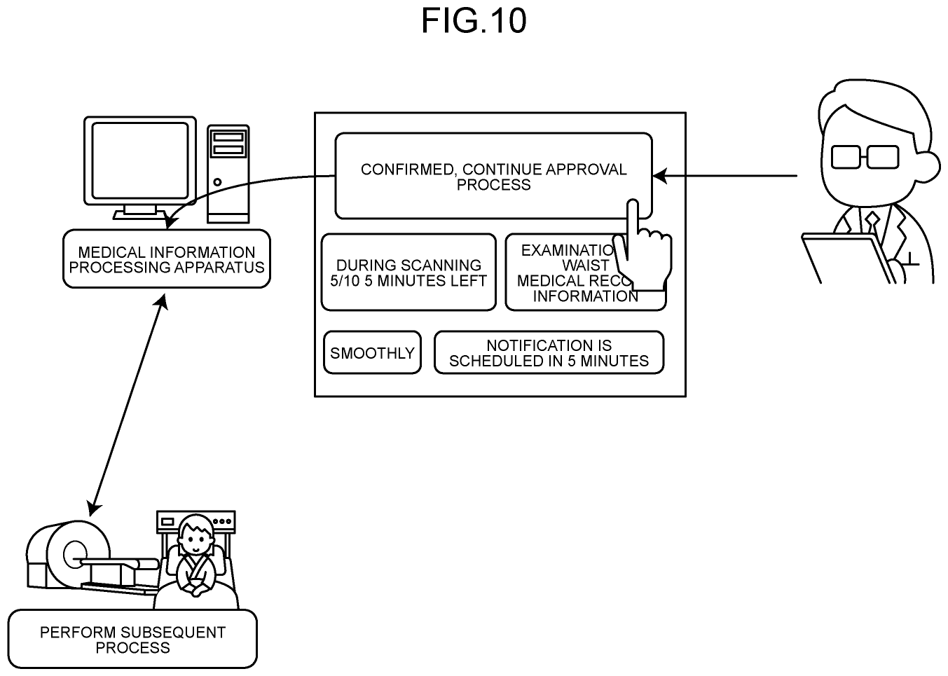

As described at step S107 of FIG. 3, the work control function 651 performs a process in response to an operation by an operator. FIG. 10 is a diagram for explaining a process according to an operation by an operator according to the first embodiment. For example, as illustrated in FIG. 10, when an operator performs an approval operation on the operation screen displayed by the control function 654, the work control function 651 controls the apparatus on the network 10 to perform a subsequent process in a workflow. In one example, when the approval operation is performed for the imaging conditions of the constructed workflow, the work control function 651 controls the medical image diagnostic apparatus 2 to perform imaging under the approved imaging conditions.

As described above, according to the first embodiment, the detection function 652 detects an event that requires an operation by an operator in a medical-related work performed by the apparatus connected to the network. The determination function 653 determines at least one operator who can respond to the event among operators who operate the terminal apparatuses 1 connected to the network. The control function 654 notifies the terminal apparatus 1 operated by the at least one operator of the occurrence of the event. Accordingly, the medical information processing apparatus 6 according to the first embodiment can request an operator who can respond to the event to respond to the event at the time the event has occurred, thereby appropriately improving the efficiency of work.

According to the first embodiment, the control function 654 causes the terminal apparatus 1 of an operator who has responded to the notification to display information on details of the event. Accordingly, the medical information processing apparatus 6 according to the first embodiment can request an operator who can respond to the event in terms of time and work to respond to the event, thereby appropriately improving the efficiency of work.

According to the first embodiment, the control function 654 causes the terminal apparatus 1 of the operator who has responded to the notification to display an operation screen for performing an operation on the event. Accordingly, the medical information processing apparatus 6 according to the first embodiment enables efficient work on the event.

According to the first embodiment, the control function 654 changes display details of the operation screen according to the attributes of the operator who has responded to the notification. Accordingly, the medical information processing apparatus 6 according to the first embodiment enables the display of an appropriate operation screen according to the operator who has responded to the notification.

According to the first embodiment, the control function 654 causes the terminal apparatuses 1 operated by operators other than the operator who has responded to the notification, among the terminal apparatuses 1 notified of the occurrence of the event, to transition to a state in which it is identifiable that the response to the notification has been executed. Accordingly, the medical information processing apparatus 6 according to the first embodiment enables appropriate control to be performed for operators who have not responded to the notification.

According to the first embodiment, the control function 654 stops the notification to the terminal apparatuses 1 operated by the operators other than the operator who has responded to the notification. Accordingly, the medical information processing apparatus 6 according to the first embodiment allows a notification regarding a responded event to be turned off.

According to the first embodiment, on the basis of the authority information and the ability information of operators who operate the terminal apparatuses 1 connected to the network 10, the determination function 653 determines an operator who can respond to the event. Accordingly, the medical information processing apparatus 6 according to the first embodiment makes it possible to appropriately determine an operator who can respond to the event.

According to the first embodiment, the work control function 651 causes the apparatus connected to the network 10 to perform a medical-related work and controls the progress of the work. Accordingly, the medical information processing apparatus 6 according to the first embodiment enables a workflow to be automatically controlled.

### Other Embodiments

The above-described embodiment has described the case in which the medical information processing apparatus 6 performs the work control function 651. However, the embodiment is not limited thereto, and the medical information processing apparatus 6 may not perform the work control function 651. That is, the medical information processing apparatus 6 may not perform the construction of a workflow and the execution and management of the workflow.

The above-described embodiment has described the case in which an operator who has responded to a notification of the occurrence of an event responds to the event. However, the embodiment is not limited thereto, and information on details of the event may be displayed on the terminal apparatus 1 of the operator who has responded to the notification of the occurrence of the event, and whether to respond to the event may be determined after the information is confirmed.

In such a case, the control function 654 causes the terminal apparatus 1 of the operator who has responded to the notification of the occurrence of the event to display information on details of the event, and to display a GUI for inputting whether to respond to the event. This allows the operator to determine whether to respond to the event after confirming details of the event.

The above-described embodiment has described a physician as an example of an operator. However, the embodiment is not limited thereto, and another medical professional such as a laboratory technician, a nurse, and co-medical may serve as the operator.

The above-described embodiment has described the case in which the status of a workflow can be confirmed when an event occurs. However, the embodiment is not limited thereto, and the workflow may be confirmed at time other than when the event occurs. In such a case, for example, the control function 654 provides the current status of the workflow in response to a confirmation request from the terminal apparatus 1.

The above-described embodiment has described the case in which an operator performs an approval or confirmation operation. However, the embodiment is not limited thereto, and a memo such as “confirmed at xx o'clock, no problem” may be input at the same time, for example.

The above-described embodiment has described the case in which a notification is made after an event occurs. However, the embodiment is not limited thereto and a notification may also be made in advance. For example, the control function 654 may give an operator advance notice that “notification is scheduled in 5 minutes”.

The above-described embodiment has described the case in which the terminal apparatus 1 performs approval of the imaging conditions and confirmation of the imaging screen. However, the embodiment is not limited thereto, and, for example, an operator may be notified to rush to a site when a problem occurs.

The above-described embodiment has described the case in which a response is made to notification. However, no response may be made to the notification. In such a case, the control function 654 performs re-notification. When the re-notification is performed, the number of notification destinations may be increased. For example, the determination function 653 may increase the number of notification destinations by changing conditions for determining the notification destinations.

The above-described embodiment has described the example in which a work control unit, a detection unit, a determination unit, and a control unit in the present specification are implemented by the work control function, the detection function, the determination function, and the control function of the processing circuitry, respectively; however, the embodiment is not limited thereto. For example, the work control unit, the detection unit, the determination unit, and the control unit in the present specification may be implemented by the work control function, the detection function, the determination function, and the control function described in the embodiment, or the same functions may also be implemented by only hardware, only software, or a combination of hardware and software.

The term “processor” used in the description of the above-described embodiments means, for example, circuitry such as a central processing unit (CPU), a graphics processing unit (GPU), an application specific integrated circuit (ASIC), and a programmable logic device (for example, a simple programmable logic device (SPLD), a complex programmable logic device (CPLD), and a field programmable gate array (FPGA)). Instead of storing computer programs in the storage circuitry, the computer programs can be configured to be incorporated directly into the circuitry of the processor. In this case, the processor implements the functions by reading and executing the computer programs incorporated in the circuitry. Each processor of the present embodiment is not limited to being configured as a single piece of circuitry for each processor, and one processor may be configured by combining a plurality of pieces of independent circuitry to implement the functions thereof.

The medical information processing program to be executed by the processor is provided by being incorporated in advance in a read-only memory (ROM), storage circuity, or the like. Note that the medical information processing program may be provided by being recorded on a computer readable non-transitory storage medium, such as a CD (compact disc)-ROM, a flexible disk (FD), a CD-R (recordable), and a digital versatile disc (DVD), in a file format installable or executable in the devices. The medical information processing program may also be provided or distributed by being stored on a computer connected to a network such as the Internet and downloaded over the network. For example, the medical information processing program is configured as a module including the above-described processing functions. As actual hardware, the CPU reads and executes the medical information processing program from the storage medium such as ROM, and each module is loaded onto a main storage device and generated on the main storage device.

In the embodiments and variations described above, each component of each apparatus illustrated in the drawings is a functional concept, and does not necessarily have to be physically configured as illustrated in the drawings. That is, the specific form of distribution or integration of each apparatus is not limited to that illustrated in the drawings, and the apparatuses can be configured by functionally or physically distributing or integrating all or some thereof in desired units according to various loads, usage conditions, and the like. Moreover, each processing function performed by each apparatus can be implemented in whole or in part by a CPU and a computer program that is analyzed and executed by the CPU, or by hardware using wired logic.

Of the processes described in the above embodiments and variations, all or some of the processes described as being performed automatically can be performed manually, or all or some of the processes described as being performed manually can be performed automatically by known methods. Other information including processing procedures, control procedures, specific names, and various data and parameters indicated in the document and drawings may be changed as desired, unless otherwise noted.

According to at least one of the embodiments described above, the efficiency of work can be appropriately improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to
detect an event that requires an operation by an operator in a medical-related work performed by an apparatus connected to a network,
determine at least one operator who is able to respond to the event among operators who operate terminal apparatuses connected to the network, and
notify the terminal apparatus operated by the at least one operator of an occurrence of the event.

2. The medical information processing apparatus according to claim 1, wherein the processing circuitry is configured to cause the terminal apparatus of the operator who has responded to the notification to display information on details of the event.

3. The medical information processing apparatus according to claim 2, wherein the processing circuitry is configured to cause the terminal apparatus of the operator who has responded to the notification to display an operation screen for performing an operation on the event.

4. The medical information processing apparatus according to claim 3, wherein the processing circuitry is configured to change display details of the operation screen according to attributes of the operator who has responded to the notification.

5. The medical information processing apparatus according to claim 1, wherein the processing circuitry is configured to cause a terminal apparatus operated by an operator other than the operator who has responded to the notification, among terminal apparatuses notified of the occurrence of the event, to transition to a state in which it is identifiable that the response to the notification has been executed.

6. The medical information processing apparatus according to claim 5, wherein the processing circuitry is configured to stop the notification to the terminal apparatus operated by the operator other than the operator who has responded to the notification.

7. The medical information processing apparatus according to claim 1, wherein, based on authority information and ability information of the operators who operate the terminal apparatuses connected to the network, the processing circuitry is configured to determine an operator who is able to respond to the event.

8. The medical information processing apparatus according to claim 1, wherein the processing circuitry is configured to cause the apparatus connected to the network to perform the medical-related work and control a progress of the work.

9. A medical information processing method comprising:
detecting an event that requires an operation by an operator in a medical-related work performed by an apparatus connected to a network;
determining at least one operator who is able to respond to the event among operators who operate terminal apparatuses connected to the network; and
notifying the terminal apparatus operated by the at least one operator of an occurrence of the event.

10. A non-transitory computer readable medium comprising instructions that cause a computer to execute:
detecting an event that requires an operation by an operator in a medical-related work performed by an apparatus connected to a network;
determining at least one operator who is able to respond to the event among operators who operate terminal apparatuses connected to the network; and
notifying the terminal apparatus operated by the at least one operator of an occurrence of the event.
